(19) United States
(12) Patent Application Publication
(10) Pub. No.: US 2023/0095345 A1
(43) Pub. Date: Mar. 30, 2023

(54) NON-CONTACT SYSTEMS AND METHODS FOR MONITORING AND ADDRESSING BREATHING EVENTS IN NEONATES

(71) Applicant: Covidien LP, Mansfield, MA (US)

(72) Inventors: Philip C. SMIT, Hamilton (GB); Paul S. ADDISON, Edinburgh (GB); Dean MONTGOMERY, Edinburgh (GB)

(21) Appl. No.: 17/814,901

(22) Filed: Jul. 26, 2022

Related U.S. Application Data
(60) Provisional application No. 63/250,839, filed on Sep. 30, 2021.

(51) Int. Cl. A61B 5/00

(52) U.S. Cl. CPC A61B 5/0022; A61B 5/0064; A61B 5/0077

(57) ABSTRACT

System and methods for video-based neonatal patient monitoring are described. Methods for neonatal patient monitoring can generally include use of a non-contact detector, such as a depth-sensing camera, to obtain data pertaining to the neonate that can then be used to calculate or otherwise determine a neonate patient breathing parameter, such as respiratory volume. The method further includes monitoring the breathing parameter to identify the occurrence of a neonate breathing event, such as apnea, and initiating a neonate stimulation event when a breathing event is identified. The stimulation event can include, e.g., vibration, auditory signals, visual signals, and/or other types of tactile signals. The systems and methods can use additional monitoring apparatus to monitor additional neonate health parameters, and incorporate this additional information into the decision of when a stimulation event should be initiated.

### CROSS-REFERENCE TO RELATED APPLICATIONS

The present application claims benefit of priority to U.S. Provisional Patent Application No. 63/250,839, entitled “NON-CONTACT SYSTEMS AND METHODS FOR MONITORING AND ADDRESSING BREATHING EVENTS IN NEONATES” and filed on Sep. 30, 2021, which is specifically incorporated by reference herein for all that it discloses or teaches.

### BACKGROUND

In preterm infants, immature respiratory control plays a role in the initiation of apnea, but the occurrence of accompanying intermittent hypoxemia may be enhanced by increased metabolic oxygen consumption and poor respiratory function (i.e., decreased oxygen uptake, pulmonary oxygen stores, and total blood oxygen carrying capacity). Periods of apnea can cause damage to the infant's developing brain and other organs. Furthermore, apnea arousal failure has been proposed as a cause for sudden death during sleep in preterm infants.

Neonatal apnea can also be relatively benign. For example, an apnea may not be associated with any detrimental physiological effect such as a desaturation. However, critical apneas in neonates is of great concern. Critical apneas are generally those associated with significant desaturation events.

Accordingly, automated systems and methods capable of identifying apnea events, including critical apnea events, and addressing such events would be useful for reducing the number, duration and/or depth of severe apnea events in neonates.

### SUMMARY

This Summary is provided to introduce a selection of concepts in a simplified form that are further described below in the Detailed Description. This Summary, and the foregoing Background, is not intended to identify key aspects or essential aspects of the claimed subject matter. Moreover, this Summary is not intended for use as an aid in determining the scope of the claimed subject matter.

In some embodiments, a video-based neonatal patient monitoring method generally includes: a step of calculating a neonatal patient breathing parameter from data obtained from a non-contact detector, the non-contact detector being aligned with the neonatal patient such that at least a portion of the neonatal patient is within a field of view of the non-contact detector; a step of monitoring the calculated neonatal patient breathing parameter to identify the occurrence of a breathing event; and a step of initiating a neonatal patient stimulation when a breathing event is identified. In some embodiments, the non-contact detector is a camera, such as a depth sensing camera, and the neonatal patient breathing parameter is respiratory volume. Neonatal patient stimulation can include, for example, vibration, an auditory signal, a visual signal, and/or a tactile signal.

In some embodiments, a video-based neonatal patient monitoring method generally includes: a step of calculating a neonatal patient breathing parameter from data obtained from a non-contact detector, the non-contact detector being aligned with the neonatal patient such that at least a portion of the neonatal patient is within a field of view of the non-contact detector; a step of monitoring the oxygen saturation level of the neonatal patient to identify the occurrence of a significant desaturation event; a step of monitoring the calculated neonatal patient breathing parameter to identify the occurrence of a breathing event; and a step of initiating a neonatal patient stimulation or an alarm when both a breathing event and a significant desaturation event are identified. In some embodiments, the non-contact detector is a camera, such as a depth sensing camera, and the neonatal patient breathing parameter is respiratory volume. Neonatal patient stimulation can include, for example, vibration, an auditory signal, a visual signal, and/or a tactile signal.

These and other aspects of the technology described herein will be apparent after consideration of the Detailed Description and Figures herein. It is to be understood, however, that the scope of the claimed subject matter shall be determined by the claims as issued and not by whether given subject matter addresses any or all issues noted in the Background or includes any features or aspects recited in the Summary.

### BRIEF DESCRIPTION OF THE DRAWINGS

Non-limiting and non-exhaustive embodiments of the disclosed technology, including the preferred embodiment, are described with reference to the following figures, wherein like reference numerals refer to like parts throughout the various views unless otherwise specified.

FIG. 1 is a schematic view of a video-based patient monitoring system configured in accordance with various embodiments of the presently described technology.

FIG. 2 is a block diagram illustrating a video-based patient monitoring system having a computing device, a server, and one or more image capturing devices, and configured in accordance with various embodiments of the presently described technology.

FIG. 3 is a simplified schematic view of a video-based patient monitoring system for neonatal patients configured in accordance with various embodiments of the presently described technology.

FIGS. 4A and 4B are illustration of a video-based patient monitory system display configured in accordance with various embodiments of the presently described technology.

FIG. 5 is a flow diagram illustrating a video-based patient monitoring method in accordance with various embodiments of the presently described technology.

FIG. 6 is a flow diagram illustrating a video-based patient monitoring method in accordance with various embodiments of the presently described technology.

FIG. 7 is a simplified schematic view of a video-based patient monitoring system for neonatal patients configured in accordance with various embodiments of the presently described technology.

FIG. 8 is an illustration of a measured oxygen saturation display configured in accordance with various embodiments of the presently described technology.

FIG. 9 is a flow diagram illustrating a video-based patient monitoring method in accordance with various embodiments of the presently described technology.

FIG. 10 is an illustration of a video-based patient monitory system display configured in accordance with various embodiments of the presently described technology.

FIG. 11 is a flow diagram illustrating a video-based patient monitoring method in accordance with various embodiments of the presently described technology.

### DETAILED DESCRIPTION

Embodiments are described more fully below with reference to the accompanying Figures, which form a part hereof and show, by way of illustration, specific exemplary embodiments. These embodiments are disclosed in sufficient detail to enable those skilled in the art to practice the invention. However, embodiments may be implemented in many different forms and should not be construed as being limited to the embodiments set forth herein. The following detailed description is, therefore, not to be taken in a limiting sense.

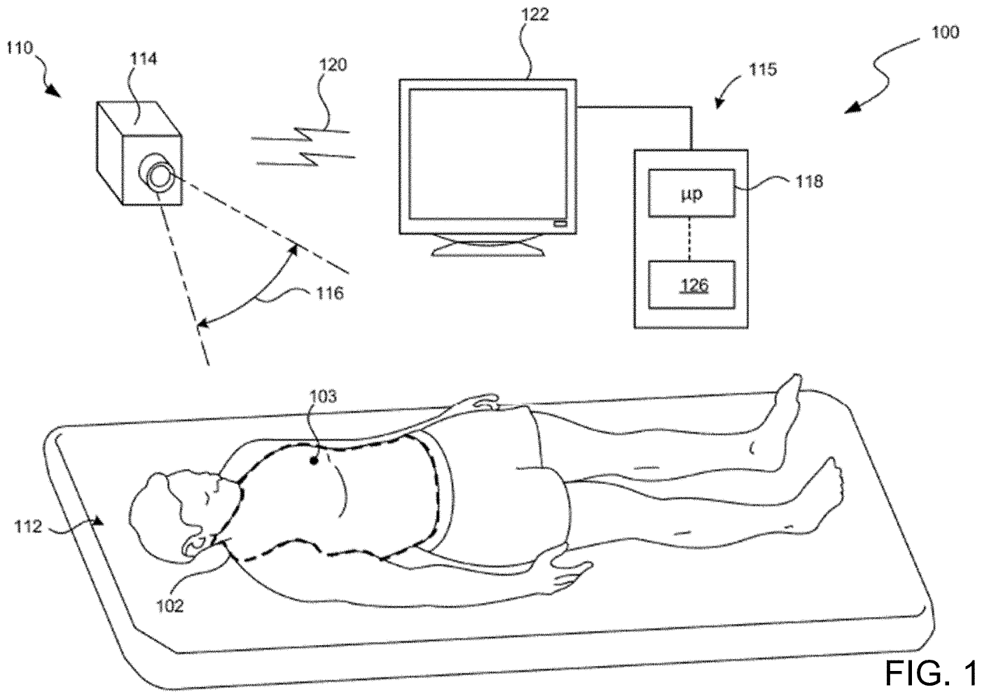

FIG. 1 is a schematic view of a patient 112 and a video-based patient monitoring system 100 configured in accordance with various embodiments of the present technology. The system 100 includes a non-contact detector 110 and a computing device 115. In some embodiments, the detector 110 can include one or more image capture devices, such as one or more video cameras. In the illustrated embodiment, the non-contact detector 110 includes a video camera 114. The non-contact detector 110 of the system 100 is placed remote from the patient 112. More specifically, the video camera 114 of the non-contact detector 110 is positioned remote from the patient 112 in that it is spaced apart from and does not contact the patient 112. The camera 114 includes a detector exposed to a field of view (FOV) 116 that encompasses at least a portion of the patient 112. While FIG. 1 illustrates an adult patient 112, it should be appreciated that the system 100 described herein can also be used in situations where the patient 112 is a child or an infant, including situations where an infant patient is inside of an isolette, incubator or crib.

The camera 114 can capture a sequence of images over time. The camera 114 can be a depth sensing camera, such as a Kinect camera from Microsoft Corp. (Redmond, Wash.) or Intel camera such as the RealSense D415, D435, and SR305 cameras from Intel Corp, (Santa Clara, Calif.). A depth sensing camera can detect a distance between the camera and objects within its field of view. Such information can be used to determine that a patient 112 is within the FOV 116 of the camera 114 and/or to determine one or more regions of interest (ROI) to monitor on the patient 112. Once a ROI is identified, the ROI can be monitored over time, and the changes in depth of regions (e.g., pixels) within the ROI 102 can represent movements of the patient 112 associated with breathing. As described in greater detail in U.S. Patent Application Publication No. 2019/0209046, those movements, or changes of regions within the ROI 102, can be used to determine various breathing parameters, such as tidal volume, minute volume, respiratory rate, respiratory, etc. Those movements, or changes of regions within the ROI 102, can also be used to detect various breathing abnormalities, as discussed in greater detail in U.S. Patent Application Publication No. 2020/0046302. The various breathing abnormalities can include, for example, low flow, apnea, rapid breathing (tachypnea), slow breathing, intermittent or irregular breathing, shallow breathing, obstructed and/or impaired breathing, and others. U.S. Patent Application Publication Nos. 2019/0209046 and 2020/0046302 are incorporated herein by reference in their entirety.

In some embodiments, the system 100 determines a skeleton-like outline of the patient 112 to identify a point or points from which to extrapolate a ROI. For example, a skeleton-like outline can be used to find a center point of a chest, shoulder points, waist points, and/or any other points on a body of the patient 112. These points can be used to determine one or more ROIs. For example, a ROI 102 can be defined by filling in an area around a center point 103 of the chest, as shown in FIG. 1. Certain determined points can define an outer edge of the ROI 102, such as shoulder points. In other embodiments, instead of using a skeleton, other points are used to establish a ROI. For example, a face can be recognized, and a chest area inferred in proportion and spatial relation to the face. In other embodiments, a reference point of a patient's chest can be obtained (e.g., through a previous 3-D scan of the patient), and the reference point can be registered with a current 3-D scan of the patient. In these and other embodiments, the system 100 can define a ROI around a point using parts of the patient 112 that are within a range of depths from the camera 114. In other words, once the system 100 determines a point from which to extrapolate a ROI, the system 100 can utilize depth information from the depth sensing camera 114 to fill out the ROI. For example, if the point 103 on the chest is selected, parts of the patient 112 around the point 103 that are a similar depth from the camera 114 as the point 103 are used to determine the ROI 102.

In another example, the patient 112 can wear specially configured clothing (not shown) that includes one or more features to indicate points on the body of the patient 112, such as the patient's shoulders and/or the center of the patient's chest. The one or more features can include visually encoded message (e.g., bar code, QR code, etc.), and/or brightly colored shapes that contrast with the rest of the patient's clothing. In these and other embodiments, the one or more features can include one or more active emitters that are configured to indicate their positions by transmitting light or other information to the camera 114. In these and still other embodiments, the one or more features can include a grid or another identifiable pattern to aid the system 100 in recognizing the patient 112 and/or the patient's movement. In some embodiments, the one or more features can be stuck on the clothing using a fastening mechanism such as adhesive, a pin, etc. For example, a small sticker can be placed on a patient's shoulders and/or on the center of the patient's chest that can be easily identified within an image captured by the camera 114. The system 100 can recognize the one or more features on the patient's clothing to identify specific points on the body of the patient 112. In turn, the system 100 can use these points to recognize the patient 112 and/or to define a ROI.

In some embodiments, the system 100 can receive user input to identify a starting point for defining a ROI. For example, an image can be reproduced on a display 122 of the system 100, allowing a user of the system 100 to select a patient 112 for monitoring (which can be helpful where multiple objects are within the FOV 116 of the camera 114) and/or allowing the user to select a point on the patient 112 from which a ROI can be determined (such as the point 103 on the chest of the patient 112). In other embodiments, other methods for identifying a patient 112, identifying points on the patient 112, and/or defining one or more ROI's can be used.

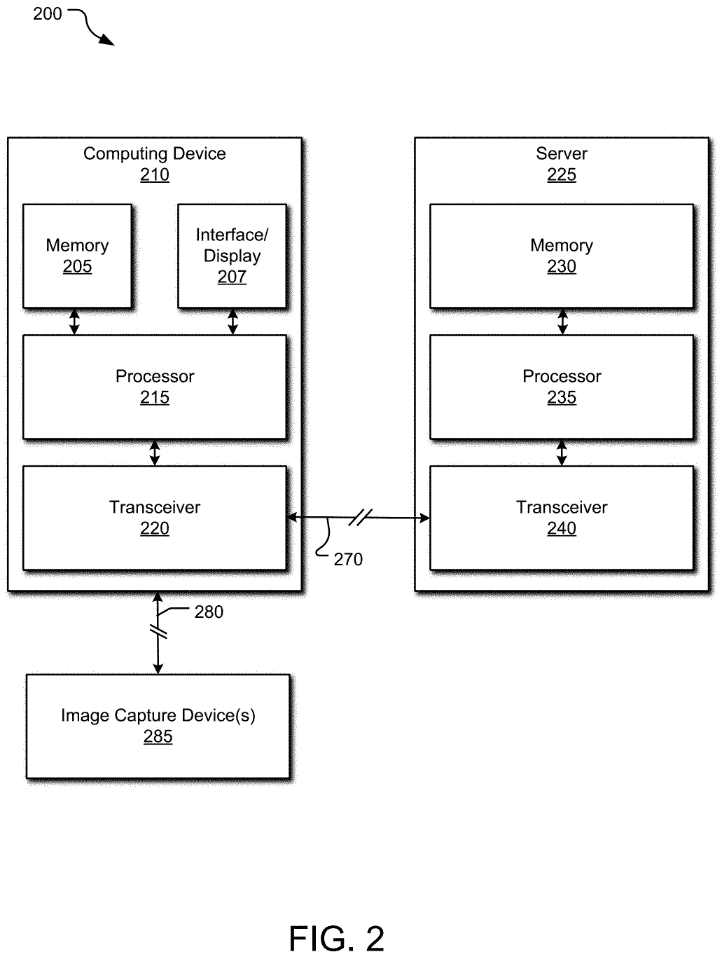

The images detected by the camera 114 can be sent to the computing device 115 through a wired or wireless connection 120. The computing device 115 can include a processor 118 (e.g., a microprocessor), the display 122, and/or hardware memory 126 for storing software and computer instructions. Sequential image frames of the patient 112 are recorded by the video camera 114 and sent to the processor 118 for analysis. The display 122 can be remote from the camera 114, such as a video screen positioned separately from the processor 118 and the memory 126. Other embodiments of the computing device 115 can have different, fewer, or additional components than shown in FIG. 1. In some embodiments, the computing device 115 can be a server. In other embodiments, the computing device 115 of FIG. 1 can be additionally connected to a server (e.g., as shown in FIG. 2 and discussed in greater detail below). The captured images/video can be processed or analyzed at the computing device 115 and/or a server to determine a variety of parameters (e.g., tidal volume, minute volume, respiratory rate, etc.) of a patient's breathing. In some embodiments, some or all of the processing may be performed by the camera, such as by a processor integrated into the camera or when some or all of the computing device 115 is incorporated into the camera.

FIG. 2 is a block diagram illustrating a video-based patient monitoring system 200 (e.g., the video-based patient monitoring system 100 shown in FIG. 1) having a computing device 210, a server 225, and one or more image capture devices 285, and configured in accordance with various embodiments of the present technology. In various embodiments, fewer, additional, and/or different components can be used in the system 200. The computing device 210 includes a processor 215 that is coupled to a memory 205. The processor 215 can store and recall data and applications in the memory 205, including applications that process information and send commands/signals according to any of the methods disclosed herein. The processor 215 can also (i) display objects, applications, data, etc. on an interface/display 207 and/or (ii) receive inputs through the interface/display 207. As shown, the processor 215 is also coupled to a transceiver 220.

The computing device 210 can communicate with other devices, such as the server 225 and/or the image capture device(s) 285 via (e.g., wired or wireless) connections 270 and/or 280, respectively. For example, the computing device 210 can send to the server 225 information determined about a patient from images captured by the image capture device(s) 285. The computing device 210 can be the computing device 115 of FIG. 1. Accordingly, the computing device 210 can be located remotely from the image capture device(s) 285, or it can be local and close to the image capture device(s) 285 (e.g., in the same room). In various embodiments disclosed herein, the processor 215 of the computing device 210 can perform the steps disclosed herein. In other embodiments, the steps can be performed on a processor 235 of the server 225. In some embodiments, the various steps and methods disclosed herein can be performed by both of the processors 215 and 235. In some embodiments, certain steps can be performed by the processor 215 while others are performed by the processor 235. In some embodiments, information determined by the processor 215 can be sent to the server 225 for storage and/or further processing.

In some embodiments, the image capture device(s) 285 are remote sensing device(s), such as depth sensing video camera(s), as described above with respect to FIG. 1. In some embodiments, the image capture device(s) 285 can be or include some other type(s) of device(s), such as proximity sensors or proximity sensor arrays, heat or infrared sensors/cameras, sound/acoustic or radio wave emitters/detectors, or other devices that include a field of view and can be used to monitor the location and/or characteristics of a patient or a region of interest (ROI) on the patient. Body imaging technology can also be utilized according to the methods disclosed herein. For example, backscatter x-ray or millimeter wave scanning technology can be utilized to scan a patient, which can be used to define and/or monitor a ROI. Advantageously, such technologies can be able to “see” through clothing, bedding, or other materials while giving an accurate representation of the patient's skin. This can allow for more accurate measurements, particularly if the patient is wearing baggy clothing or is under bedding. The image capture device(s) 285 can be described as local because they are relatively close in proximity to a patient such that at least a part of a patient is within the field of view of the image capture device(s) 285. In some embodiments, the image capture device(s) 285 can be adjustable to ensure that the patient is captured in the field of view. For example, the image capture device(s) 285 can be physically movable, can have a changeable orientation (such as by rotating or panning), and/or can be capable of changing a focus, zoom, or other characteristic to allow the image capture device(s) 285 to adequately capture images of a patient and/or a ROI of the patient. In various embodiments, for example, the image capture device(s) 285 can focus on a ROI, zoom in on the ROI, center the ROI within a field of view by moving the image capture device(s) 285, or otherwise adjust the field of view to allow for better and/or more accurate tracking/measurement of the ROI.

The server 225 includes a processor 235 that is coupled to a memory 230. The processor 235 can store and recall data and applications in the memory 230. The processor 235 is also coupled to a transceiver 240. In some embodiments, the processor 235, and subsequently the server 225, can communicate with other devices, such as the computing device 210 through the connection 270.

The devices shown in the illustrative embodiment can be utilized in various ways. For example, either the connections 270 and 280 can be varied. Either of the connections 270 and 280 can be a hard-wired connection. A hard-wired connection can involve connecting the devices through a USB (universal serial bus) port, serial port, parallel port, or other type of wired connection that can facilitate the transfer of data and information between a processor of a device and a second processor of a second device. In another embodiment, either of the connections 270 and 280 can be a dock where one device can plug into another device. In other embodiments, either of the connections 270 and 280 can be a wireless connection. These connections can take the form of any sort of wireless connection, including, but not limited to, Bluetooth connectivity, Wi-Fi connectivity, infrared, visible light, radio frequency (RF) signals, or other wireless protocols/methods. For example, other possible modes of wireless communication can include near-field communications, such as passive radio-frequency identification (RFID) and active RFID technologies. RFID and similar near-field communications can allow the various devices to communicate in short range when they are placed proximate to one another. In yet another embodiment, the various devices can connect through an internet (or other network) connection. That is, either of the connections 270 and 280 can represent several different computing devices and network components that allow the various devices to communicate through the internet, either through a hard-wired or wireless connection. Either of the connections 270 and 280 can also be a combination of several modes of connection.

The configuration of the devices in FIG. 2 is merely one physical system 200 on which the disclosed embodiments can be executed. Other configurations of the devices shown can exist to practice the disclosed embodiments. Further, configurations of additional or fewer devices than the devices shown in FIG. 2 can exist to practice the disclosed embodiments. Additionally, the devices shown in FIG. 2 can be combined to allow for fewer devices than shown or can be separated such that more than the three devices exist in a system. It will be appreciated that many various combinations of computing devices can execute the methods and systems disclosed herein. Examples of such computing devices can include other types of medical devices and sensors, infrared cameras/detectors, night vision cameras/detectors, other types of cameras, augmented reality goggles, virtual reality goggles, mixed reality goggle, radio frequency transmitters/receivers, smart phones, personal computers, servers, laptop computers, tablets, blackberries, RFID enabled devices, smart watch or wearables, or any combinations of such devices.

Referring back to FIG. 1, the display 122 can be used to display various information regarding the patient 112 monitored by the system 100. In some embodiments, the system 100, including the video camera 114, the computing device 115 and the processor 118, is used to obtain depth measurements and use those depth measurements to calculate respiratory volume values as described in greater detail in U.S. Patent Application Publication No. 2019/0209046. The calculated respiratory volume values can then be displayed on the display 122, such as on a graph displayed on the display 122 and in which the respiratory volume value is displayed as a function of time via a plot line.

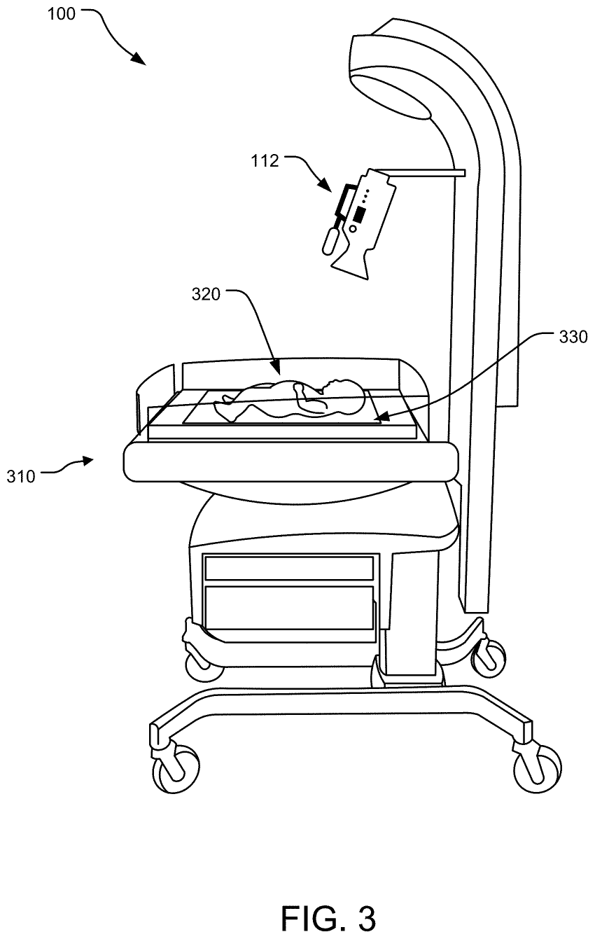

FIG. 3 is a simplified version of the system 100 shown in FIG. 1 and in which the system 100 is used in a neonatal setting. More specifically, the system 100 (including camera 112) is installed proximate an isolette, incubator, or crib 310 (hereinafter generally referred to as an incubator 310) in which a neonatal patient 320 resides. As shown in FIG. 3, the camera 112 is positioned such that at least a portion of the neonatal patient 320 is within the field of view of the camera 112. In this regard, the camera 112 is able to obtain data relating to the neonatal patient 310, such as depth data in embodiments where the camera 112 is a depth-sensing camera. This depth data can then be used to calculate various neonatal patient breathing parameters, such as a respiratory volume signal.

As also shown in FIG. 3, the incubator 310 may be furnished with a stimulation device 330, which is integrated into system 100 such that the stimulation device 330 may be automatically initiated and terminated by the system 100 based on the occurrence of various events being monitored by the system 100. In FIG. 3, the stimulation device 330 is shown as being a vibration element positioned under the neonate 320. Accordingly, the vibration element may be initiated by the system 100 upon the occurrence of specified events being monitored by the system 100 to thereby vibrate the neonate 320. In one embodiment, which is discussed in greater detail below, the system 100 is configured to determine when the neonate 320 is experiencing an apnea event, at which point the system 100 initiates the vibration element. The aim of the vibration element is to stimulate the neonate 320 and encourage it to restart normal breathing.

While FIG. 3 generally illustrates the stimulation device 330 as being a vibration element located under the neonate 320, it should be appreciated that the specific type of stimulation device 330 used in connection with system 100, as well as the specific location of the stimulation device 300, is generally not limited. For example, when the stimulation device 330 is a vibration element, the vibration element may be a pad laid over the incubator mattress and on top of which the neonate resides, the vibration element may be incorporated into/inside the mattress on which the neonate 320 resides, or the vibration element may be attached to the walls or other part of the incubator so as to vibrate the entire incubator upon initiation. Other types of stimulation devices that can be used include, but are not limited to, an auditory signal (e.g., a speaker located anywhere on or within the incubator), a visual signal (e.g., a flashing light located anywhere on or within the incubator), or a tactile signal (e.g., a jet or nozzle located anywhere on or within the incubator and capable of expelling puffs or a stream of air directed at the neonate).

The incubator 310 may include one type of stimulation device 330, or multiple types of stimulation devices. As discussed in greater detail below, the use of multiple types of stimulation devices in a single incubator may allow for the system 100 to adaptively select the type of stimulation device determined to be most effective at rousing the neonate upon occurrence of a breathing event.

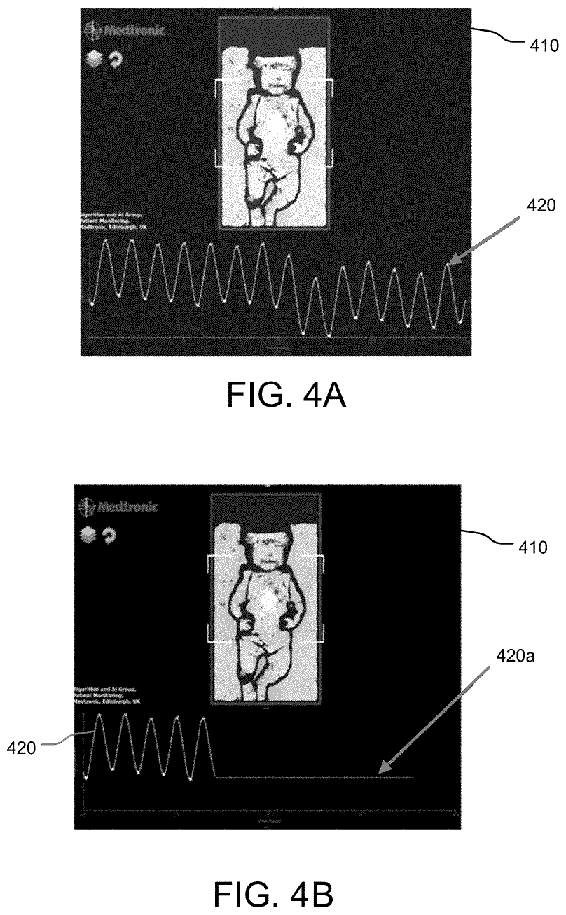

With reference to FIGS. 4A and 4B, an exemplary user interface image that can be presented on display 122 associated with system 100 provides a depth sensing image 410 of the neonate and a continuous real-time or near real-time measurement of a breathing parameter 420 calculated by the system 100 from the data obtained by the camera 112. In FIGS. 4A and 4B, the breathing parameter 420 is a respiratory volume waveform which can be obtained, e.g., through the integration of depth changes within the field of view of the depth camera. Any suitable breathing parameter 420 can be obtained/calculated by the system 100 and presented on image 410. In FIG. 4A, the respiratory volume waveform indicates normal breathing due to normal, periodic inhalation and exhalation by the neonate. In FIG. 4B, an apnea event is represented by portion 420a of the respiratory volume waveform 420, which has flattened out, thereby indicating an absence of inhalation or exhalation.

System 100, including computing device 115 and processor 118, is configured to continuously or at least periodically monitor the measured breathing parameter 420 and identify instances where the calculated data indicates the occurrence of a breathing event, such as an apnea event. Thus, in the case of FIG. 4B, the system 100 identifies the breathing event due to the cessation of inhalation and exhalation indicated by the flattened section 420a of the respiratory volume waveform 410, and initiates the appropriate action in response to the identification of the breathing event. In some embodiments, the appropriate action initiated by the system 100 upon identification of a breathing event is the initiation of one or more stimulation devices.

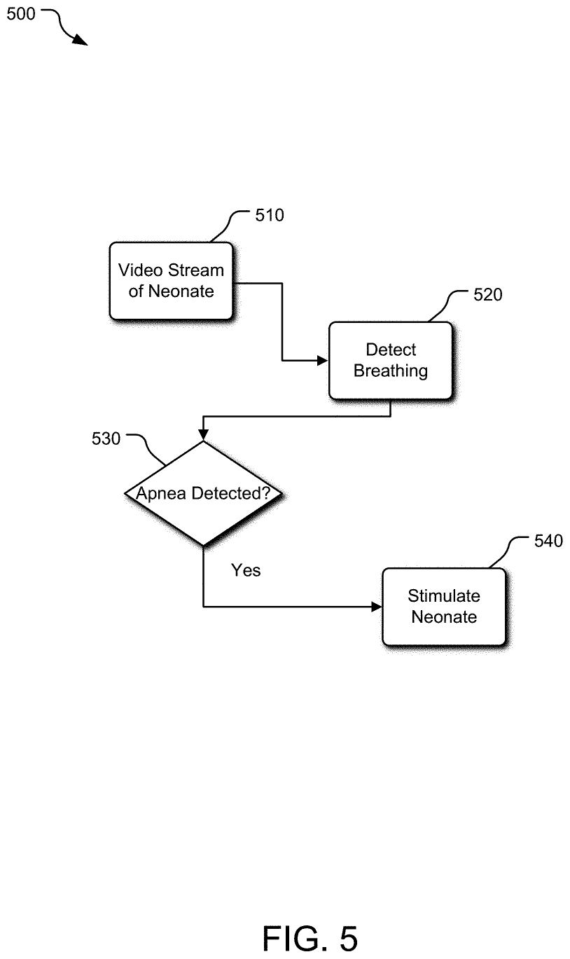

FIG. 5 illustrates a flow diagram of method 500 capable of being executed by system 100 for monitoring neonate breathing and triggering a stimulation event upon occurrence of an identified breathing event. Method 500 begins with a step 510 of obtaining a video stream of a neonate. As described previously, in some embodiments, the video stream is obtained from a depth sensing camera, though other types of cameras can be used to provide the video stream of the neonate.

In step 520, data is extracted from the video stream and used to determine or calculate a neonate breathing parameter. In embodiments where the camera is a depth sensing camera, the video stream is used to collect depth data relating to the movement of various parts of the neonate's body, which data is then used in step 520 to calculate a neonatal breathing parameter. In one example, integration of depth changes occurring within a region of interest covering a portion of the neonatal patient's body (e.g., the chest area) allows for calculation of the neonate's respiratory volume. Continuous or at least periodic calculation of this value allows for creation of a respiratory volume waveform.

In step 530, the breathing parameter being continuously or at least periodically calculated by system 100 is continuously or at least periodically monitored for occurrence of a breathing event, such as apnea. The specific breathing event or events being monitored is generally not limited, provided that the system 100 is programmed to look for and identify specific types of changes in a measured breathing parameter that are correlated to a breathing event. For example, the system 100 may be programmed to look for and identify when a respiratory volume waveform exhibits an extended plateau, since such plateaus are correlated to the cessation of inhalation or exhalation and therefore an apnea event.

When step 530 is carried out and no breathing event is detected, the method 500 continues to detect and monitor breathing at step 520 with no other actions being taken.

When step 530 is carried out and a breathing event such as an apnea event is detected, the method 500 may proceed to step 540 in which a stimulation device is initiated to stimulate the neonate. More specifically, the system 100 (having stimulation element or elements 330 integrated therewith) instructs initiation of one or more of the stimulation elements 330. As noted previously, the aim of initiating stimulation element 330 upon detection of a breathing event is to rouse or otherwise stimulate the neonate such that normal breathing resumes. As also noted previously, the specific type of stimulation element used to stimulate the neonate at step 540 is not limited. In some embodiments, such as those using the system shown in FIG. 3, the neonate is stimulated by initiating a vibration element located under the neonate. This vibration may sufficiently rouse, startle or otherwise stimulate the neonate such that normal breathing resumes.

In some embodiments, initiation of the stimulation element includes one or more instances of the specific stimulation. For example, when vibration is used as the stimulation, the vibration imitated upon detection of a breathing event can be a single instance of vibration for a set period of time (e.g., 5 second) or a series of vibrations separated by time periods of no vibration (e.g., three 5 second vibrations, each separated by 3 seconds of no vibration). In some embodiments where multiple instances of stimulation are used, the multiple instances are non-patterned, non-periodic sequences such that the neonate does not get use to the stimulation and learn to ignore it. When a series of stimulation events are used, each stimulation event can also vary in duration and/or intensity. For example, with vibration, the first vibration may for a relatively short period of time at a relatively gentle vibration, while subsequent vibrations in the series may increase in duration and/or intensity. When an auditory stimulation is used, the volume of the auditory stimulation may increase between a first auditory stimulation and subsequent auditory stimulations in the series.

While not expressly illustrated in FIG. 5, the method 500 may include continuous monitoring of patient breathing or the patient breathing parameter, even after a breathing event such as apnea is detected. In this manner, the system 100 and method 500 may further identify when a breathing event has ended, i.e., when normal breathing resumes following a breathing event. In such embodiments, the system 100 may terminate the stimulation event after it has determined that the breathing event has ended. In some embodiments, the stimulation event initiated upon identification of a breathing event continues until the breathing event is identified as being over, while in other embodiments, the stimulation event is one or a defined series of stimulation events that terminates upon completion of the series regardless of whether the system identifies the breathing event as having ended.

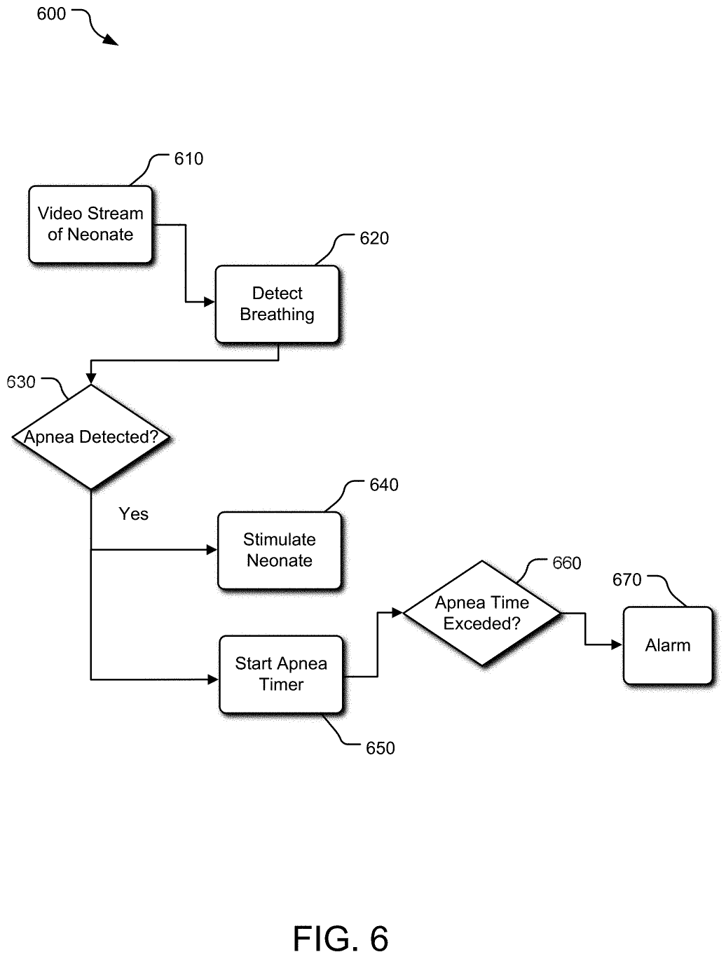

Various additional elements or features may be added to the method shown in FIG. 5 to improve and/or expand upon the monitoring and stimulation methods described herein. FIG. 6 illustrates a method 600 that builds upon method 500, specifically by tracking the duration of a breathing event and taking additional steps when the breathing event extends for longer than a predetermined amount of time. Method 600 includes steps 610, 620, 630, 640 that are similar or identical to previously described steps 510, 520, 530, 540, respectively. Thus, method 600 begins by obtaining a video stream of the neonate in step 610, detecting a breathing parameter in step 620, detecting a breathing event in step 630, and stimulating the neonate in step 640 when a breathing event is detected in step 640. Method 600 further includes starting a breathing event time at step 650 when a breathing event is detected in step 630. The system 100 continuously or at least periodically continues to monitor the neonate's breathing to determine if/when the breathing event has terminated, while also comparing the duration of the breathing event against a predetermined time period at step 660. If the duration of the breathing event is determined to have exceeded the predetermined time period at 660, then the system proceeds to sound an alarm at step 670. Alternatively, if the breathing event is determined to have ended and the duration of the terminated breathing event is determined to be less than the predetermined time period at step 660, then no alarm is initiated, the breathing event timer is reset to zero, and the system continues to monitor the neonate's breathing to identify if/when another breathing event occurs.

The type of alarm used in step 670 is generally not limited. In some embodiments, the alarm may be an audible or visual alarm that is located at or near the isolette, incubator or crib to thereby warn caretakers in the vicinity of the neonate. In some embodiments, the alarm is an alert sent to a caregiver's computer, phone, tablet or other handheld device such that regardless of where the caregiver is relative to the neonate's location, the caregiver is alerted to the prolonged breathing event. In such situations, the alarm or alert may be provided in the form a text, email, automated phone call, or other message type that is sent to the caregiver.

Variations of method 600 can include other types of actions initiated by the system once it is determined that the breathing event has exceeded the predetermined period of time. For example, instead of initiating an alarm at step 670, step 670 may entail increasing the intensity and/or duration of the stimulation event initially started at step 640, repeating the stimulation event first carried out at step 640 (in embodiments where the stimulation event is one or a series of stimulation events that are initially only performed once), or even changing the type of stimulation event or adding another stimulation event. For example, if the initial stimulation event performed at step 640 is a vibration stimulation, but the duration of the breathing event continues past the predetermined time period, then step 670 may entail adding an auditory or visual stimulation event to go with the vibration stimulation.

In some embodiments, the system 100 is programmed such that the camera 112 and associated processing equipment is capable of determining whether a caregiver is present with the neonate, such as identifying when a caregiver is located within the field of view of the neonate. Any suitable manner of making this determination can be used by the system, including the use of artificial intelligence and machine learning. In such embodiments where the system 100 includes the ability to identify when a caregiver is present with the neonate, the system 100 may further include programming such that stimulation events and/or alarms are not initiated when a breathing event is detected. In such embodiments, methods 500 and 600, for example, would be prohibited from performing steps 540, 640 (i.e., initiating the stimulation element) if a caregiver is identified as being present with the neonate, even if a breathing event is detected at step 530, 630.

In some embodiments, the system 100 further includes the ability to record various information regarding the response of the neonate to stimulation events and adaptively change the manner in which the system 100 performs in the future based on the previously recorded information. Such embodiments may require the system 100 to include machine learning and/or artificial intelligence, although simpler algorithms may also be used.

Non-limiting examples of information that may be recorded for use in adaptively changing the performance of system 100 includes each instance of a detected breathing event, the type of neonatal patient stimulation initiated in response to each breathing event, whether the breathing event was terminated in response to the specific type of neonatal stimulation initiated, the number of detected breathing events, and the duration of each detected breathing event. Using this data, the system 100 can be programmed to adaptively change the manner in which a future detected breathing event is handled based on the success or failure of previous attempts to handle a detected breathing event.

In one example of this adaptive modification, the system 100 monitors the number of breathing events that have previously occurred, including optionally the number of breathing events that have previously occurred within a predetermined period of time. If the number of detected breathing events exceeds a predetermined number, or if the number of breathing events in a set period of time exceeds a predetermined number, then the system 100 may adaptively change such that an alarm is immediately sounded for any subsequently detected breathing events, rather than first attempting a stimulation event to try and terminate the breathing event. Such a change in the operation of system 100 may be useful when the number of breathing events that has occurred is so great as to denote a more serious health condition or situations so as to require immediate caretaker intervention rather than first attempting stimulation intervention.

In a related example of the previously described adaptive modification, the system 100 tracks both the number of previously detected breathing events, and whether a stimulation event was successful in terminating the breathing event. In such embodiments, if the number of times that a breathing event is detected and not successfully terminated by a stimulation event, then the system 100 may adaptively change such that subsequently identified breathing events are responded to by bypassing a stimulation event and instead proceeding directly to initiation of an alarm.

In still a further example of an adaptive modification, the system 100 tracks each occurrence of a breathing event, which type of stimulation event was used in response to each breathing event, and whether or not the specific type of stimulation event was successful in terminating the breathing event. In such embodiments, if the number of times a specific type of stimulation event is used and does not terminate the breathing event exceeds a predetermined number, then the system 100 can adaptively change to no longer use this type of stimulation event for future detected breathing events. Similarly, if the number of times a specific type of stimulation event is used and successfully terminates the breathing event is exceeded, the system 100 can adaptively change to exclusively use this type of stimulation event for future detected breathing events. Variations on this adaptive method can also be used with respect to different patterns of stimulation used within a specific type of stimulation. For example, if a first vibration pattern is found to be more successful at terminating a breathing event than a second vibration pattern, the system 100 can adapt to use only the first vibration pattern for subsequently detected breathing events.

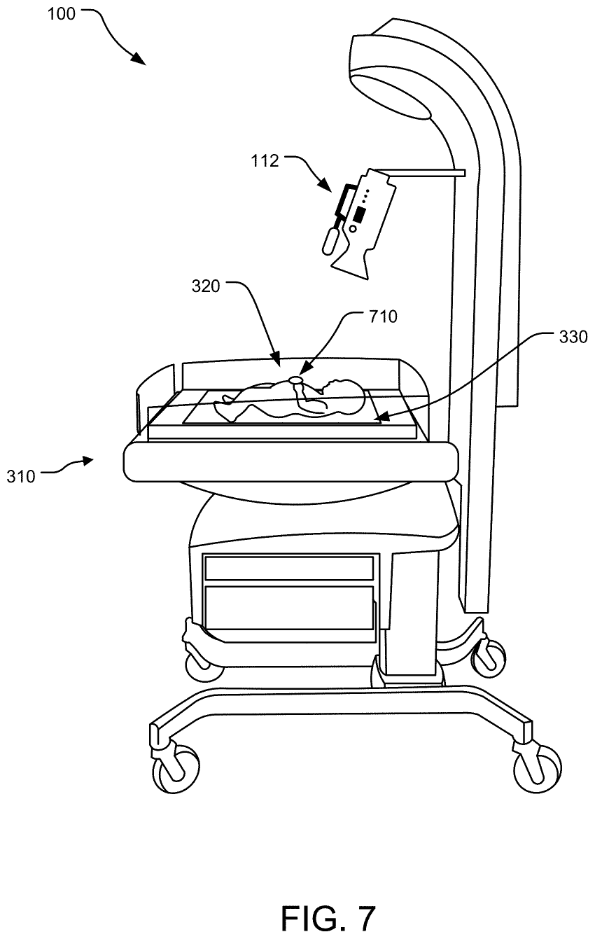

Additional monitoring elements can be added to the system to assist in improved identification of breathing events, including identifying breathing events that are considered to be critical breathing events. In some embodiments, a pulse oximeter is integrated into the system 100 to monitor oxygen saturation in conjunction with the other breathing parameter or parameters monitored by the system via a non-contact detector such as a depth sensing camera. An embodiment of this configuration is shown in FIG. 7, wherein system 100 is similar to the system 100 shown in FIG. 3, but further includes a pulse oximeter 710 attached to the neonate 320 residing in incubator 310. The pulse oximeter 710 measures the oxygen salutation of neonate 320 and transmit measured oxygen saturation data to the system 100 for processing and display as discussed in further detail below. The system 100 also continues to obtain data from the camera 112 (e.g., depth data where camera 112 is a depth sensing camera) so that the system can also calculate and monitor a breathing parameter such as respiratory volume of the neonate 320. As discussed in greater detail below, both parameters (i.e., oxygen saturation and respiratory volume) are then used to determine when to initiate a stimulation element 330 (e.g., vibration element) and/or alarm.

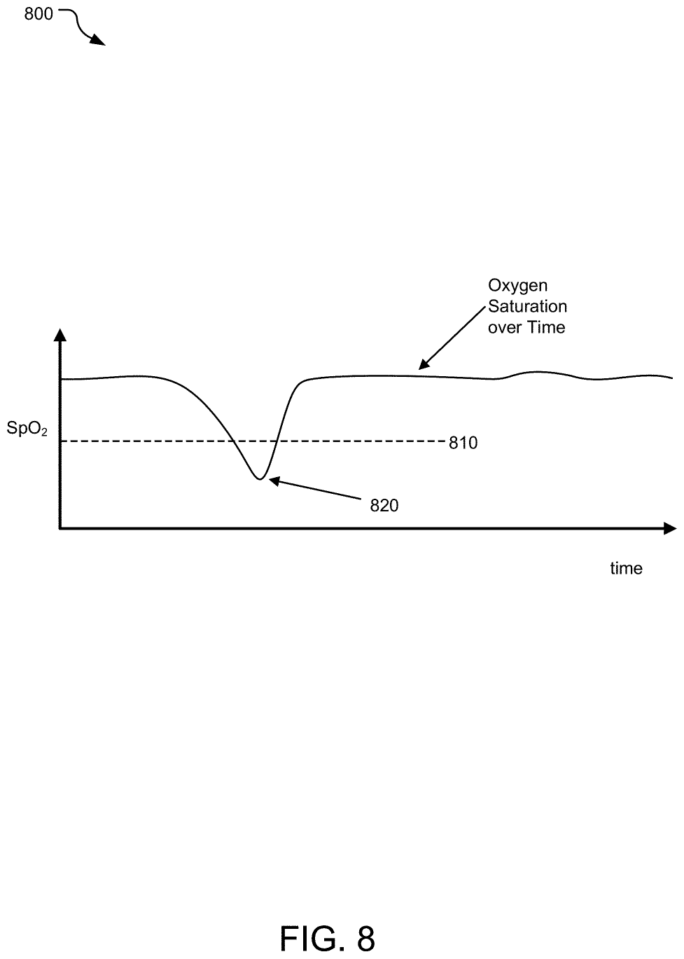

FIG. 8 is an illustration of how the measured oxygen saturation data obtained from pulse oximeter 710 can be tracked over time and displayed on, for example, display 122 of system 100. As shown in FIG. 8, a threshold value 810 is established (e.g., programmed within system 100), the threshold value 810 setting a value below which significant oxygen desaturation is considered to have occurred. The threshold value can be any value set by, e.g., the caregiver, and is adjustable based on, e.g., different patients or monitoring conditions. In some embodiments, a significant oxygen desaturation event is considered to have occurred when a greater than 5%, 10%, 15%, 20%, etc., drop is oxygen saturation occurs, and the threshold value can be set based on this definition. As shown in FIG. 8, a significant oxygen desaturation event 820 has been identified in the monitored oxygen saturation of the neonate due to the measured oxygen saturation value falling below the threshold value 810.

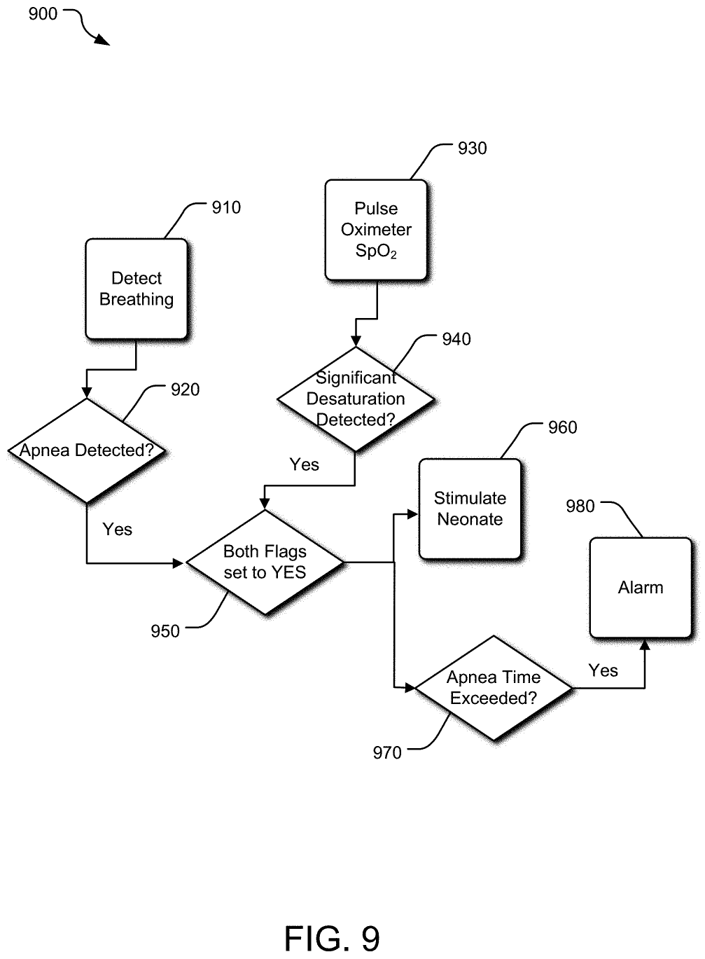

Having now established methods and systems for tracking at least two different patient health parameters of the neonate (e.g., a breathing parameter such as respiratory volume obtained from a depth sensing camera, and oxygen saturation obtained from a pulse oximeter), the system 100 can be configured such that initiation of a stimulation event and/or other alarming is determined based upon both patient health parameters. FIG. 9 illustrates such a method 900, wherein stimulation of the neonate is initiated only upon confirmation that the neonate is experiencing both a breathing event (e.g., apnea) and a significant desaturation event. As shown in FIG. 9, method 900 generally includes a step 910 of monitoring a neonate breathing parameter, such as respiratory volume. As described in greater detail below, this breathing parameter can be obtained by first using a depth sensing camera to obtain depth data relating to, e.g., the movement of a neonate's chest, and then using this data to calculate a respiratory volume waveform. This breathing parameter is then monitored and checked at step 920 to determine whether a breathing event is occurring, such as an apnea event (as can be indicated by, e.g., the plateauing of the respiratory volume waveform). When a breathing event is identified at step 920, then a first flag is raised to yes at step 950.

Method 900 further includes step 930 of monitoring a neonate's oxygen saturation using, e.g., a pulse oximeter attached to the neonate as shown above in FIG. 7. The neonate's oxygen saturation level is then monitored and checked at step 940 to determine whether a significant oxygen desaturation event, such as the significant oxygen desaturation event 820 shown in FIG. 8, is occurring. When a significant oxygen desaturation event is identified at step 940, then a second flag is raised to yes at step 950.

When both the first and second flag are raised to yes at step 950, the method proceeds to step 960, wherein a stimulation event is initiated to try and stimulate the neonate for the purpose of rousing the neonate and encouraging the resumption of normal breathing. As described in greater detail above with respect to FIG. 5, any stimulation event or any combination of stimulation events can be used. Furthermore, the method 900 may incorporate any of the adaptive modifications described previously wherein the system 100 implementing method 900 modifies they type, pattern, intensity, etc. of the stimulation based on previously recorded effectiveness information and learning regarding the most effective stimulations for rousing the neonate. While not shown expressly in FIG. 9, when only one or no flags are raised at step 950, no stimulation is initiated, and the system continues to monitor the neonate's breathing parameter and oxygen saturation.

FIG. 9 further illustrates method 900 including a step 970 of monitoring the duration of the breathing event (e.g., apnea event) and determining whether the duration of the breathing event exceeds a predetermined length of time. If the predetermined length of time is exceeded, the method proceeds to step 980 wherein an alarm is initiated. The initiated alarm can be similar or identical to the alarm described in greater detail above with respect to FIG. 6. While step 970 is shown in FIG. 9 as being directed to monitoring and identifying the occurrence of a breathing event, it should be appreciated that step 970 could also be used to monitor the length of a significant oxygen desaturation event. Step 970 could also monitor the duration of both the breathing event and the significant oxygen desaturation event and only initiate an alarm at step 980 when both events exceed a predetermined length of time (which may be the same length of time, or distinct lengths of time for each type of event).

In an alternative embodiment to the method 900 shown in FIG. 9, the method 900 is truncated such that an alarm is immediately initiated at step 960 rather than initiating a stimulation event. Such a modification may be desirable in situations where the simultaneous or near simultaneous occurrence of both a breathing event and significant oxygen desaturation event is considered indicative of a critical apnea event. That is to say, identifying the occurrence of a significant oxygen desaturation event at the same time as an apnea event may serve as confirmation that the urgency of the apnea event should be raised from a non-critical apnea event to a critical apnea event. A critical apnea event may be considered an apnea event requiring, e.g., immediate caregiver attention and intervention, as opposed to only automatic initiation of a stimulation event without caregiver intervention.

The method 900 of FIG. 9 may further be modified such that non-critical and critical alarms are initiated earlier in the method and at different check points in the method 900. For example, method 900 may be modified to include the initiation of a non-critical alarm at the detection of a breathing event at step 920 and/or at the detection of a significant oxygen desaturation event at step 940, and the initiation of a critical alarm at step 960 after it has been confirmed at step 950 that both a breathing event and a significant oxygen desaturation event are occurring simultaneously or near simultaneously. Non-critical alarms and critical alarms can be different in, for example, tone, volume, length, etc., to provide caregivers with a clear indication as to whether an alarm is a non-critical or critical alarm.

While FIG. 9 can be interpreted as illustrating a method wherein monitoring for a breathing event such as apnea and monitoring for a significant desaturation event occur in parallel, it should be appreciated that FIG. 9 can also be considered as disclosing a method wherein the two separate monitoring events are occurring in series. For example, the method 900 shown in FIG. 9 can begin by monitoring a breathing parameter such as respiratory volume at step 920 and identifying the occurrence of a breathing event such as apnea at step 920, and only after a breathing event is identified at step 920 does the method check whether a significant desaturation event is occurring at step 940. Similarly, the method 900 can begin by monitoring oxygen saturation at step 930 and identifying the occurrence of a significant desaturation event at step 940, and only after a significant desaturation event is identified at step 940 does the method check whether a breathing event is occurring at step 920.

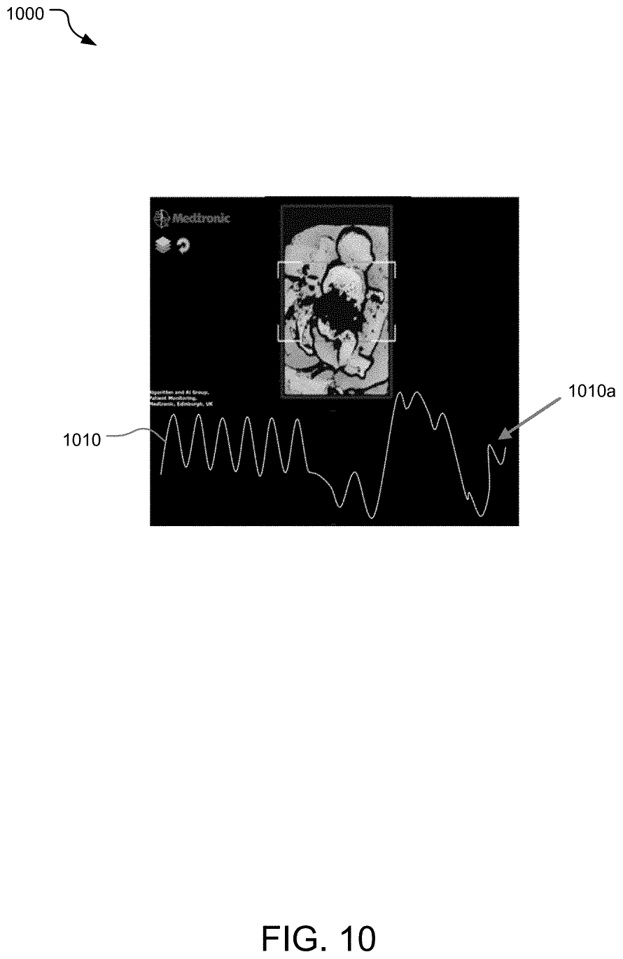

With reference back to FIGS. 4A, 4B, and 8, exemplary displays for real time or near real time monitoring of respiratory volume and oxygen saturation levels illustrate instances where the monitored neonate is generally still or exhibiting little motion. However, in many real-world applications, the monitored neonate will not be motionless. In such scenarios, the display for both respiratory volume and oxygen saturation level can be distorted in such a way that may provide a false positive for a breathing event (e.g., apnea) or significant oxygen desaturation event. For example, FIG. 10 provides an illustration of a respiratory volume waveform 1010 wherein neonate motion is occurring at segment 1010a, thus disrupting the normal periodic waveform generally used to confirm normal neonate breathing. Despite the neonate still possibly exhibiting normal breathing at segment 1010a, the system 100 may improperly identify segment 1010a as indicating an apnea event, and therefore initiate a stimulation and/or alarm component of the overall monitoring method. Similar disruptions in a measured oxygen desaturation waveform may also occur during neonate motion, which can lead to a false positive identification of a significant oxygen desaturation event. Accordingly, the system and methods described herein may require incorporation of safeguards against such false positives.

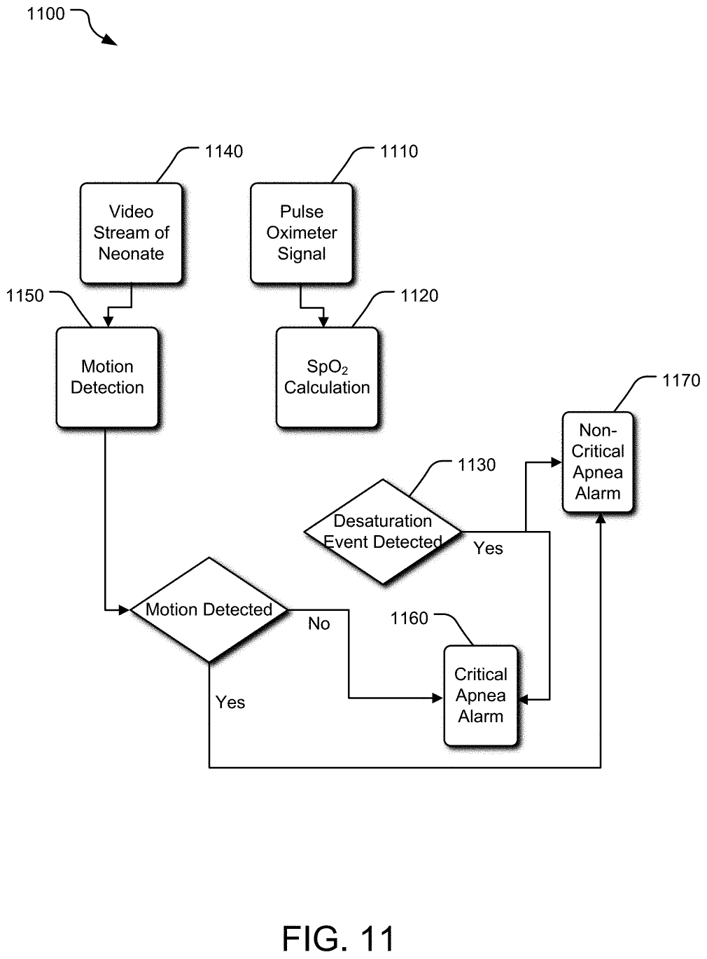

In a variation on method 900 shown in FIG. 9, the overall method is modified to incorporate a motion detection component associated with one or more monitoring and identification phases of method 900. For example, and as described in greater detail below with respect to FIG. 11, steps 930 and 940 of method 900 may be complemented with motion detection steps to help ensure that the identification of a saturation event is not the result of a false positive cause by neonatal motion.

With reference now to FIG. 11, method 1100 generally includes a step 1110 of obtaining a pulse oximeter signal, such as through the use of a pulse oximeter attached the neonate and which is incorporated into the system 100 such that the pulse oximeter signal obtained in step 1110 can be received and processed by the system 100; and a step 1120 of calculating the oxygen saturation level for the neonate using the pulse oximeter signal obtained in step 1110. Steps 1110 and 1120 generally overlap with step 930 shown in FIG. 9. In step 1130, the neonate's measured oxygen saturation level is monitored to identify a desaturation event. Step 1130 is therefore similar or identical to step 940 described above, such as checking to determine if/when the oxygen saturation level falls below a predetermined threshold value.

In the method shown in FIG. 9, the identification of a desaturation event at step 940 together with the identification of an apnea event at step 920 can lead to the initiation of a critical alarm based on the determination that the simultaneous occurrence of these two events likely denotes a critical apnea event. FIG. 11, however, illustrates an embodiment where determination of a desaturation event with the identification of an apnea event (not shown in FIG. 11) will lead to the initiation of either a critical alarm or a non-critical alarm. The decision of whether a critical or non-critical alarm is initiated is dependent on an additional check that is conducted to ensure that the determination of a desaturation event is not a false positive caused by neonate motion. While FIG. 11 focuses on running this motion check against the determination of a desaturation event, the method could be modified such that motion check is run against the determination of an apnea event, or the check could be run against both events prior to deciding which type of alarm to initiate.

In order to complete this check, method 1100 further includes a step 1140 of obtaining a video stream of the monitored neonate (step 1140 being similar or identical to previously described methods of using a camera 112 to obtain a video stream of a neonate) and a step 1150 of analyzing the video stream to detect neonate motion. Any known methods for identifying neonate movement in the video stream can be used. In the scenario where 1) an apnea event is identified (not shown in FIG. 11), 2) a desaturation event is identified at step 1130, and 3) it is confirmed that there is no neonate motion corresponding to the time of the identified desaturation event, then step 1160 of initiating a critical alarm is performed. Alternatively, in the scenario where 1) an apnea event is identified (not shown in FIG. 11), 2) a desaturation event is identified at step 1130, and 3) it is confirmed that there is neonate motion corresponding to the time of the identified desaturation event, then step 1170 of initiating a non-critical alarm is performed. The basis for initiating a non-critical alarm when motion is identified at the same time as the desaturation event is that the identification of the desaturation event may be a false positive due to neonate motion, and therefore the two conditions for a critical apnea event (i.e., both an apnea event and a desaturation event) have not been met. However, a non-critical alarm is still initiated in this scenario such that identifications made by the system are not wholly ignored.

In a modification to the method shown in FIG. 11, the duration of the neonate motion detected at step 1150 is measured. If the desaturation event detected at step 1130 persists and the duration of the motion detected at step 1150 exceeds a predetermined length of the time, then the non-critical alarm initially performed at step 1170 (due to the initial detection of neonate motion coinciding with the desaturation event) is converted to a different alarm than the non-critical alarm to notify the caretaker of the prolonged motion and desaturation event. In some embodiments, the switch in alarm may even be from a non-critical alarm to a critical alarm.

While many of the embodiments described herein have focused on the use of a depth sensing camera and the use of depth data obtained therefrom to calculate a patient breathing parameter, it should be appreciated that the systems and methods described herein are not limited to the use of depth sensing cameras and depth data. Any other type of cameras suitable for collecting a type of data from which a breathing parameter can be calculated or otherwise obtained can also be used in the systems and methods described herein. For example, the camera 112 in system 100 could be an RGB, IR, thermal, or any other type of camera.

From the foregoing, it will be appreciated that specific embodiments of the invention have been described herein for purposes of illustration, but that various modifications may be made without deviating from the scope of the invention. Accordingly, the invention is not limited except as by the appended claims.

Although the technology has been described in language that is specific to certain structures and materials, it is to be understood that the invention defined in the appended claims is not necessarily limited to the specific structures and materials described. Rather, the specific aspects are described as forms of implementing the claimed invention.

Because many embodiments of the invention can be practiced without departing from the spirit and scope of the invention, the invention resides in the claims hereinafter appended.

Unless otherwise indicated, all number or expressions, such as those expressing dimensions, physical characteristics, etc., used in the specification (other than the claims) are understood as modified in all instances by the term “approximately”. At the very least, and not as an attempt to limit the application of the doctrine of equivalents to the claims, each numerical parameter recited in the specification or claims which is modified by the term “approximately” should at least be construed in light of the number of recited significant digits and by applying rounding techniques. Moreover, all ranges disclosed herein are to be understood to encompass and provide support for claims that recite any and all sub-ranges or any and all individual values subsumed therein. For example, a stated range of 1 to 10 should be considered to include and provide support for claims that recite any and all sub-ranges or individual values that are between and/or inclusive of the minimum value of 1 and the maximum value of 10; that is, all sub-ranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

I/we claim:

1. A video-based neonatal patient monitoring method, comprising:
calculating a neonatal patient breathing parameter from data obtained from a non-contact detector, the non-contact detector being aligned with the neonatal patient such that at least a portion of the neonatal patient is within a field of view of the non-contact detector;
monitoring the calculated neonatal patient breathing parameter to identify the occurrence of a breathing event; and
initiating a neonatal patient stimulation when a breathing event is identified.

2. The video-based neonatal patient monitoring method of claim 1, wherein the non-contact detector is a depth sensing camera and the data obtained from the non-contact detector is depth measurements.

3. The video-based neonatal patient monitoring method of claim 1, wherein the neonatal breathing parameter is respiratory volume.

4. The video-based neonatal patient monitoring method of claim 1, wherein the breathing event is apnea.

5. The video-based neonatal patient monitoring method of claim 1, wherein the neonatal patient stimulation is one or more of: vibration, an auditory signal, a visual signal, or puffs of air stimulation.

6. The video-based neonatal patient monitoring method of claim 1, further comprising:
when a breathing event has been identified, monitoring the calculated neonatal patient breathing parameter to identify when the breathing event has terminated.

7. The video-based neonatal patient monitoring method of claim 6, further comprising:
terminating the neonatal patient stimulation when the breathing event has terminated.

8. The video-based neonatal patient monitoring method of claim 6, further comprising:
initiating an alarm when the breathing event has not terminated after a predetermined amount of time following the start of the breathing event.

9. The video-based neonatal patient monitoring method of claim 6, further comprising:
increasing the intensity and/or frequency of the neonatal patient stimulation when the breathing event has not terminated after a predetermined amount of time following the start of the breathing event.

10. The video-based neonatal patient monitoring method of claim 1, further comprising:
determining if a caregiver is present in the field of view of the non-contact detector; and
preventing initiation of the neonatal patient stimulation when a caregiver is determined to be in the field of view of the non-contact detector.

11. The video-based neonatal patient monitoring method of claim 6, further comprising:
recording data pertaining to each instance of a breathing event, the type of neonatal patient stimulation initiated in response to the breathing event, and whether the breathing event was terminated in response to the type of neonatal stimulation initiated; and
for subsequently identified breathing events, adaptively initiating the type of neonatal patient stimulation determined to be most effective in terminating the breathing event based on the previously recorded data.

12. The video-based neonatal patient monitoring method of claim 6, further comprising:
recording data pertaining to each instance of a breathing event, the type of neonatal patient stimulation initiated in response to the breathing event, and whether the breathing event was terminated in response to the type of neonatal stimulation initiated; and
for subsequently identified breathing events, immediately initiating an alarm in lieu of initiating the neonatal patient stimulation when a predetermined number of previously recorded instances of breathing events have not been terminated by the neonatal patient stimulation.

13. The video-based neonatal monitoring method of claim 1, wherein the neonatal patient stimulation comprises a series of stimulation events, and the series of stimulation events is non-periodic.

14. A video-based neonatal patient monitoring method, comprising:
calculating a neonatal patient breathing parameter from data obtained from a non-contact detector, the non-contact detector being aligned with the neonatal patient such that at least a portion of the neonatal patient is within a field of view of the non-contact detector;
monitoring the oxygen saturation level of the neonatal patient to identify the occurrence of a significant desaturation event;
monitoring the calculated neonatal patient breathing parameter to identify the occurrence of a breathing event; and
initiating a neonatal patient stimulation or an alarm when both a breathing event and a significant desaturation event are identified.

15. The video-based neonatal patient monitoring method of claim 14, further comprising:
measuring the duration of the breathing event; and
initiating an alarm if the duration of the breathing event exceeds a predetermined length of time.

16. The video-based neonatal patient monitoring method of claim 14, further comprising:
via the non-contact detector, determining if the neonatal patient is moving when a breathing event is identified;
via the non-contact detector, determining if the neonatal patient is moving when a significant desaturation event is identified;
initiating a non-critical alarm when patient movement is occurring during either the breathing event or the significant desaturation event; and
initiating a critical alarm when patient movement is not occurring during both the breathing event and the significant desaturation event.

17. The video-based neonatal patient monitoring method of claim 16, wherein the non-critical alarm is changed to a critical alarm when the duration of the significant desaturation event exceeds a predetermined length of time.

18. The video-based neonatal patient monitoring method of claim 14, wherein monitoring the oxygen saturation level of the neonatal patient to identify the occurrence of a significant desaturation event is performed only after the occurrence of a breathing event has been identified.

19. The video-based neonatal patient monitoring method of claim 14, wherein monitoring the calculated neonatal patient breathing parameter to identify the occurrence of a breathing event is performed only after the occurrence of a significant desaturation event has been identified.

20. The video-based neonatal patient monitoring method of claim 14, wherein the non-contact detector comprises a depth-sensing camera and the breathing event comprises apnea.

21. The video-based neonatal patient monitoring method of claim 14, wherein the oxygen saturation level of the neonatal patient is obtained using a pulse oximeter attached to the neonatal patient.

22. The video-based neonatal patient monitoring method of claim 14, wherein the neonatal patient stimulation is one or more of: vibration, an auditory signal, a visual signal, or puffs of air stimulation.
